(19) United States
(12) Patent Application Publication
(10) Pub. No.: US 2005/0106243 A1
(43) Pub. Date: May 19, 2005

(54) Process for the preparation of free-flowing, pulverized atorvastatin adsorbates

(76) Inventors: Karl-Heinz Doser, Buchholz l. d. Nordheide (DE); Klaus Glaenzer, Hamburg (DE); Robert Waldraff, Memmingen (DE)

Correspondence Address: J C PATENTS, INC., 4 VENTURE, SUITE 250, IRVINE, CA, 92618, US

(21) Appl. No.: 10/990,723

(22) Filed: Nov. 16, 2004

(30) Foreign Application Priority Data
Nov. 18, 2003 (EP) 03026546.6

(51) Int. Cl. A61K 31/724; A61K 31/401; A61K 9/20

(52) U.S. Cl. 424/464; 514/423; 514/58

(57) ABSTRACT

The invention relates to a method for the preparation of atorvastatin adsorbates and solvates thereof, wherein one starts from a solution comprising the pharmaceutical active pharmaceutical ingredient substantially dissolved therein, one suspenses an adsorber material therein selected from the group of the celluloses, cellulose derivatives, polyols, sugars, sugar derivatives, maltodextrins, cyclodextrins, starches, polydextroses or mixtures thereof, and one removes the solvent by drying. Also, the invention relates to atorvastatin adsorbates obtainable according to this method as well as pharmaceutical formulations comprising them.

This application claims the benefit of priority European patent application no. 03 026 546.6-2114 filed Nov. 18, 2003, which is incorporated herein by reference in its entirety for all purposes.

### BACKGROUND OF THE INVENTION

The present invention relates to a novel method for the preparation of atorvastatin adsorbates and hydrates or solvates thereof, respectively. In an especially preferred embodiment the atorvastatin adsorbates according to the present invention comprise the active pharmaceutical ingredient in a finely dispersed amorphous form. According to the invention it is especially preferred that the active pharmaceutical ingredient is an alkaline earth metal salt, especially a hemicalcium salt as well as hydrate forms thereof.

Further, the invention relates to atorvastatin adsorbates and hydrates or solvates thereof, respectively, which are obtainable according to the afore-mentioned method.

Finally, the invention also relates to pharmaceutical formulations for the preparation of which the afore-mentioned atorvastatin adsorbates are used. Preferred drug forms according to the invention are tablets, capsules, pellets and granulates which are produced by usual pharmaceutically acceptable adjuvants in a manner known in itself. Tablets which rapidly release the active pharmaceutical ingredient and which are produced by direct pressing of the atorvastatin adsorbates according to the invention are especially preferred according to the invention.

The active pharmaceutical ingredient known as the INN atorvastatin is also known by the chemist as calcium salt of [R-(R*,R*)]-2-(4-fluorophenyl)-β,δ-dihydroxy-5-(1-methylethyl)-3-phenylphenylamino)carbonyl]-1H-pyrrol-1-heptanoic acid. This active pharmaceutical ingredient is an excellent inhibitor of the enzyme 3-hydroxy-3-methylglutaryl-(coenzyme A)-reductase I, also known by the acronyme HMG-CoA-reductase; thus it is usable as hypolipidemic and hypocholesterolemic active pharmaceutical ingredient for the therapy of lipidic metabolic disorders.

Thus HMG-CoA-reductase inhibitors are used successfully for preventing and for treating coronary heart diseases and other diseases being based on arteriosclerotic vascular changes. Thereby a significant lipid reduction, especially of the cholesterol, into the plasma can be achieved by various action modes (see e.g. Milestones in Drug Therapy/HMG-CoA-Reductase Inhibitors, Ed. G. Schmitz and M. Torzewski, Birkhauser Verlag Basel-Boston-Berlin [2002]).

The chemical structure of the atorvastatin was described in its racemic form for the first time in EP 247 633. The hemicalcium salt of the active [R-(R*,R*)] form used as active pharmaceutical ingredient was disclosed for the first time in EP 409 281 and was described as solid; but this document does not include a disclosure relating to a possible crystallinity of the product.

The production of the atorvastatin and important intermediates such as the lactone precursor of the atorvastatin is mentioned in various patent specifications, e.g. in EP 330 172, EP 553 213, EP 687 263, EP 915 866, EP 1 054 860, WO 99/57109, WO 01/72706, WO 02/55519.

It is known from the state of the art that atorvastatin in form of its hemicalcium salt does not only exist as amorphous solid, as described already by EP 409 281, but can also be obtained in more than 30 crystalline polymorphous forms.

Thus WO 97/03958 as well as WO 97/03959 filed at the same time describe the crystalline forms III as well as I, II and IV of the hemicalcium salt of the atorvastatin. While the form III is obtained by incubation of the form II for 11 days in an atmosphere having a moisture content of 95%, the forms I, II and IV can all be obtained from methanol or from methanol-water mixtures in various mixing ratios and at different temperatures. Very often the desired polymorphous form according to these documents can indeed only be obtained by a massive use of seed crystals.

WO 01/36384 discloses the polymorphous form V of the atorvastatin in form of its hemicalcium salt which is also obtained from a methanol-water mixture.

Also, WO 02/57229 describes a polymorphous form V of the atorvastatin obtained by stirring a suspension of raw atorvastatin hemicalcium in a mixture of water and absolute ethanol in the ratio of 14:3. However, according to the characterizations disclosed by the documents these two forms are different.

WO 02/41834 describes the polymorphous form VII of the atorvastatin in form of its hemicalcium salt which can be obtained by stirring a suspension of the polymorphous form V or the polymorphous form I of the atorvastatin in form of its hemicalcium salt in absolute ethanol at room temperature.

WO 02/43732 discloses the polymorphous forms VI, VIII, IX, X, XI and XII of the atorvastatin in form of its hemicalcium salt, wherein the form VI can be obtained from aqueous acetone, the form VIII can be obtained from ethanol or from n-butanol, the form IX can be obtained from ethanol or from n-butarol, the form X can be obtained from aqueous ethanol, the form XI can be obtained from methyl ethyl ketone and the form XII can be obtained from aqueous ethanol. In case of similar solvents it is found that the obtainment of a certain polymorphous form is dependent on minorly changed conditions such as different temperatures or a special water content.

WO 02/51804 uses a terminology (for describing the polymorphous forms of the atorvastatin) being different to the above patent documents. It describes the polymorphous forms X, A, B1, B2, C, D and E of the atorvastatin in form of its hemicalcium salt, wherein the form X can be obtained from methanol and methyl-t-butylether, the form A can be obtained from isopropanol containing traces of water, the form B1 can be obtained from an acetonitrile-THF mixture, the form B2 can be obtained from pure acetonitrile, the form C can be obtained from aqueous isopropanol, the form D can be obtained from ethanol and the form E can be obtained from methyl ethyl ketone by precipitating by peptone. Here it is also found that minor changes in the other conditions already lead to crystallization of another polymorphous form for the same solvents.

Finally, WO 03/04470 describes the polymorphous forms V, VI, VII, VIII, IX, X, XI, XII, Xm, XIV, XV, XVI, XVII, XVIII and XIX of the atorvastatin in form of its hemicalcium salt, wherein the form V characterized as trihydrate is obtained from aqueous acetonitrile, the form VI is obtained from aqueous DMF, the form VII characterized as sesquihydrate is obtained from aqueous acetone, the form VIII being a dihydrate is again obtained from aqueous DMF, and the form IX is again obtained from aqueous acetone. The form X being a trihydrate is obtainable from aqueous isopropanol, the form XI is again obtainable from aqueous acetonitrile, the form XII is obtainable from THF containing water, the form XIII is obtainable from aqueous methanol and the form XIV being a hexahydrate is again obtainable from aqueous isopropanol. The form XV being described as trihydrate arises from aqueous acetone, the form XVI being described as tetrahydrate-acetonitrile solvate arises from aqueous acetonitrile. The form XVIII being a solvate arises from a DMF-water-acetonitrile mixture and the form XIX also being a solvate arises from methyl-ethyl-ketone. Finally, the form XVII being a tetrahydrate is obtained by drying the form XVI for a long time. Also it will be apparent again from this disclosure that minor changes of the crystallization conditions can lead to other polymorphous forms for the use of the same solvent systems. Also it is found in this patent document, as described in the above cited disclosures, for a crystallization of the atorvastatin hemicalcium salt, that the formation of defined polymorphous forms is also strongly dependent on the starting polymorph, possibly also on seed crystals and on the different drying conditions.

In summary, it is found that the crystallization of atorvastatin hemicalcium in a defined polymorphous form is extraordinarily strongly dependent on minorly changed process parameters leading to a very costly process control since such a defined polymorph has to be absolutely guaranteed for a pharmaceutically active pharmaceutical ingredient to meet the regulatory requirements for medicaments and obviously also to ensure the constant quality of the medicament, and thus, the taking security for the patients.

A possibility to solve this problem and to reach a more advantageous isolation process is the use of amorphous atorvastatin hemicalcium. For obtaining the amorphous form several methods are described in prior art, e.g. in EP 839 132: from a solution of the active pharmaceutical ingredient in a THF-toluene mixture by removing the solvent, in EP 1 185 264: from a solution of the active pharmaceutical ingredient in THF by precipitating the product by heptane or according to EP 1 235 800 by “recrystallization” of the active pharmaceutical ingredient by lower alcohols.

The use of amorphous atorvastatin hemicalcium as active pharmaceutical ingredient in pharmaceutical formulations involves on the one hand the problem of the lower stability of the amorphous form in comparison to crystalline active pharmaceutical ingredient to surrounding conditions such as oxygen, light, heat, and residual humidity, as well as the higher sensitivity in relation to stability-reducing interactions with pharmaceutical adjuvants and additives. So it is e.g. known that atorvastatin hemicalcium in the presence of humidity and slightly acid-reacting substances very easily converts into the atorvastatin lactone which can lead to a non-acceptable active pharmaceutical ingredient decrease in the finished tablet. Mostly, to prevent the influence of such reactions in the finished drug alkalinizing additives such as special alkali and alkaline earth compounds, alkaline buffering systems (see, e.g., U.S. Pat. No. 5,180,589, U.S. Pat. No. 5,686,104, WO 00/35425 and WO 01/93859) or also the use of polymeric compounds having amido or amino groups (polyvinylpyrrolidone or cholestyramine, see WO 01/76566) for stabilization have been proposed. Also, a combination of both stabilization kinds (sodium hydroxide plus polyvinylpyrrolidone) has been used for analogous statin compounds (WO 98/57917).

A further problem of the use of amorphous atorvastatin hemicalcium as active pharmaceutical ingredient is the fact that, according to the experimental experiences of the inventors of the present application, there are during the precipitation process of the amorphous form often also heterogeneous products obtained: one part crystalline and another part amorphous, which leads to the precipitation process having to be repeated. But each additional production step poses the risk of a lost of substance of approx. 5% to 10%, being certainly not desired from an economic point of view for active pharmaceutical ingredients such as atorvastatin hemicalcium which has to be produced in a lengthy, costly and expensive synthesis series.

However, there is known from DE 10008506 A1 a method for producing an active pharmaceutical ingredient granulate for an analogous statin active pharmaceutical ingredient, namely cerivastatin, avoiding such a loss of substance, wherein an exclusively aqueous active pharmaceutical ingredient solution is directly granulated in the presence of a filler and a binder, and the so-obtained granulates are further processed after drying in tablets. But for the problem existing in the present case in relation to the active pharmaceutical ingredient atorvastatin hemicalcium, it is not a technically performable solution, because the active pharmaceutical ingredient is present in a maximum amount of 0.5% (w/w) in the granulates described by DE 10008506 A1. For therapeutic dosage of the atorvastatin hemicalcium the active pharmaceutical ingredient amount has to range between 20 mg and 80 mg. This would according to the disclosure of the DE 10008506 A1 lead to tablet weights between 4 and 16 g. The range acceptable for tablets being suitable for oral use, i.e. which have to be swallowed by the patient, ranges between 100 mg and 1 g.

Therefore, the known methods for producing pharmaceutical formulations of the atorvastatin hemicalcium are, insofar they can be performed, technically very costly, lengthy and expensive and do not solve the problem of a stable drug until now or do not solve it satisfactorily. The latter especially applies to the atorvastatin hemicalcium being preferred due to its better producibility and due to good dissolution properties and it has a high specific surface, i.e., especially amorphous or finely powdered. In such cases the stabilizations of prior art being described above are not sufficient.

Therefore, it is the object of the present invention to develop a simple and cheap method for producing stable atorvastatin powder systems which can be used directly for producing pharmaceutical formulations, wherein this method is however not limited to an especially preferred active pharmaceutical ingredient morphology, and avoids the afore-mentioned disadvantages.

### SUMMARY OF THE INVENTION

Accordingly, the present invention provides a method for the preparation of atorvastatin adsorbates and solvates thereof, comprising the steps of starting from a solution of atorvastatin or one of its salts, hydrates, solvates, esters, lactones and tautomeric derivatives thereof in at least one organic solvent having a total water content of not more than 10 vol.-%, preferably not more than 5 vol.-%,
suspending therein an adsorber material selected from the group consisting of celluloses, cellulose derivatives, polyols, sugars, sugar derivatives, maltodextrins, cyclodextrins, starches,
polydextroses or mixtures thereof, and
removing the solvent by drying.

In a preferred embodiment of the invention the atorvastatin adsorbates comprise the active pharmaceutical ingredient in a finely dispersed amorphous form, especially as calcium salt. The amorphous atorvastatin according to the present invention can be both in water-free form and in form of solvates or hydrates, respectively.

Further, the invention relates to the atorvastatin adsorbates and solvates or hydrates thereof, respectively, which are obtainable by the afore-mentioned method. Further, the invention relates to pharmaceutical formulations comprising the novel atorvastatin adsorbates. The pharmaceutical formulations optionally comprise further adjuvants and can be converted into the desired administration form. Tablets rapidly releasing the active pharmaceutical ingredient and being produced by direct pressing are especially preferred.

### DETAILED DESCRIPTION OF THE INVENTION

For the inventive method for the preparation of the atorvastatin adsorbates organic solvents are suitable for the solution comprising the pharmaceutically active pharmaceutical ingredient.

Especially, the organic solvents are selected from the group of the lower alkanols having 1 to 4 carbon atoms, the group of the ethers and the group of the aliphatic ketones and mixtures of the afore-mentioned solvents. Methanol, ethanol, isoproponol, n-propanol, acetone and other solvents such as ethyl acetate, methyl ethyl ketone, MTBE (methyl-tert-butylester) and mixtures of ethyl acetate and hexane as well as mixtures of the afore-mentioned solvents are especially preferred.

Pharmaceutically acceptable adjuvants which are suitable for rapid active pharmaceutical ingredient release such as celluloses and cellulose derivatives, especially microcrystalline cellulose, polyols, especially mannitol, sugars and sugar derivatives, especially lactose, maltodextrin, starches, cyclodextrins, polydextroses or mixtures of the afore-mentioned substances are according to the invention used as absorber materials. Microcrystalline cellulose, lactose and mannitol are preferred according to the invention.

According to the present invention the ratio of the pharmaceutical active pharmaceutical ingredient and adsorber material is in the range of 1:0.1 to 10, wherein a range of 1:1 to 1:2 is especially preferred.

For the preparation of the pharmaceutical formulations, while tablets being especially preferred, all common pharmaceutical adjuvants can be used. E.g. celluloses and cellulose derivatives (e.g. microcrystalline cellulose, native cellulose, hydroxypropyl cellulose, hydroxypropyl methyl cellulose, methyl cellulose), sugars (e.g. lactose, fructose, saccharose, glucose, maltose), sugar alcohols (e.g. lactide, mannitol, sorbitol, xylitol), inorganic fillers (e.g. calcium phosphates and calcium sulfates) and starches (e.g. corn starch, potato starch, wheat starch, dextrins, pregelatinized starches) can be used as fillers. In addition, all further adjuvants such as lubricants, decomposition auxiliaries, wetting agents, alkaline additives, stabilizers as well as aromatics, colour pigments and colours being known by those skilled in the art due to their galenical basic knowledge can be used for producing the medicament formulations according to the invention.

The amount of binder in the whole mixture of the medicament preparation is preferably 0 to 20% (m/m), the amount of fillers and adjuvants in the whole mixture is 20 to 99%, preferably 50 to 99%.

Surprisingly stable adsorbates of atorvastatin, especially amorphous atorvastatin, are produced by the method according to the invention. These atorvastatin adsorbates are used as pharmaceutically active pharmaceutical ingredient in the preparations according to the invention. Preferably atorvastatin in form of its salts is used within the scope of this invention. Examples of the atorvastatin salts used according to the invention are the monosodium and the monopotassium salts as well as the magnesium and calcium salts as well as the hydrates and solvates thereof. Especially preferably are the atorvastatin hemicalcium salt and the hydrates and solvates thereof.

Further, the respective hydrates, solvates, esters, lactones and tautomeric derivatives of the atorvastatin which can especially arise within the scope of the production of the active pharmaceutical ingredient in solution can be used within the scope of this invention avoiding the isolation of the pure active pharmaceutical ingredient.

According to the invention there has been found a method starting from a solution of the atorvastatin or one of its salts, hydrates, solvates, esters, lactones and tautomeric derivatives in organic solvent and leading to active pharmaceutical ingredient absorbates which can directly be processed further.

In principle the active pharmaceutical ingredient solution of atorvastatin can be produced in an embodiment of the invention by dissolving the atorvastatin or one of its salts, hydrates, solvates, esters, lactones and tautomeric derivatives in a suitable organic solvent; however, it is advantageous to directly use the active pharmaceutical ingredient solution anyway arising within the scope of the synthesis without isolation of the atorvastatin.

For example the atorvastatin can be produced according to the EP 409 281 A1, Example 10, done then without the recrystallization step by dissolving in ethyl acetate and precipitating by hexane, and instead the adsorber material is suspended in the active pharmaceutical ingredient solution, and the solvent is later removed by drying. Then the kind of the organic solvent results in the individual case from the final synthesis step of the active pharmaceutical ingredient production.

To this organic active pharmaceutical ingredient solution a pharmaceutically acceptable adjuvant is added which is not soluble or low soluble therein as adsorber material, well wetted and the solvent is removed immediately thereafter by drying. The drying operation can be supported by temperature control and vacuum. It is advantageously performed so that a uniform distribution is done by suitable mechanical influence (rotating, staggering, stirring motion). The solvent can be recovered by working in closed system and employed again for the following process. It is a property of the invention that a precipitation and isolation of the atorvastatin is lapsed. Atorvastatin containing adsorbates produced according to the method described can be employed directly for subsequent processing to drug forms such as tablets, capsules, pellets or granulates, preferably for subsequent processing by means of a direct compression method.

Optionally the adsorbates or drug forms so obtained can further be provided with coatings of pharmaceutical polymethacrylates such as films of Eudragit®, methyl cellulose, ethyl celluloses, hydroxypropyl methyl celluloses, cellulose acetate phthalates and/or shellac for special uses to achieve a special use purpose, e.g. controlled release of active pharmaceutical ingredient, taste covering. For this purpose, there exist sufficient technical possibilities which are at the disposal for those skilled in the pharmaceutical art.

Surprisingly, it was found that the adsorbates produced according to the method of the invention bind the active pharmaceutical ingredient without forming crystal structures typical for active pharmaceutical ingredients. This could be shown by means of x-ray diffraction measurements. Additionally, comparing stability studies show that the preferred atorvastatin adsorbates have a better stability and a faster dissolution rate than the pure amorphous active pharmaceutical ingredient. Special adsorber materials, e.g. on basis of silica have stronger binding properties, and therefore another release behaviour for the adsorbed atorvastatin.

Particularly, the mentioned properties are also maintained when the atorvastatin adsorbates are processed to drug forms such as tablets.

### BRIEF DESCRIPTION OF THE DRAWINGS

The invention is further described by the following non-limiting examples which refer to the accompanying FIGS. 1 to 11, short articulars of which are given below.

FIG. 1 a powder x-ray diffraction diagram of an atorvastatin-microcrystalline cellulose adsorbate according to the invention (in the ratio of 1:1) in the upper curve as well as microcrystalline cellulose alone in the lower curve as comparison,

FIG. 2 a powder x-ray diffraction diagram of an atorvastatin-mannitol adsorbate according to the invention (in the ratio of 1:1) in the upper curve as well as mannitol alone in the lower curve as comparison,

FIG. 3 a powder x-ray diffraction diagram of an atorvastatin-lactose adsorbate according to the invention (in the ratio of 1:1) in the upper curve as well as lactose alone in the lower curve as comparison,

FIG. 4 a powder x-ray diffraction diagram of an atorvastatin-microcrystalline cellulose adsorbate according to the invention (in the ratio of 1:1.5) in the upper curve as well as microcrystalline cellulose alone in the lower curve as comparison,

FIG. 5 a powder x-ray diffraction diagram of an atorvastatin-mannitol adsorbate according to the invention (in the ratio of 1:1) in the upper curve as well as mannitol alone in the lower curve as comparison,

FIG. 6 a powder x-ray diffraction diagram of an atorvastatin-microcrystalline cellulose adsorbate according to the invention (in the ratio of 1:2) in the upper curve as well as microcrystalline cellulose alone in the lower curve as comparison,

FIG. 7 a powder x-ray diffraction diagram of crystalline atorvastatin (so-called form I, cf. WO 97/03959 A1) in powder mixture ratio with microcrystalline cellulose of 1:1 as well as microcrystalline cellulose alone in the lower curve as comparison,

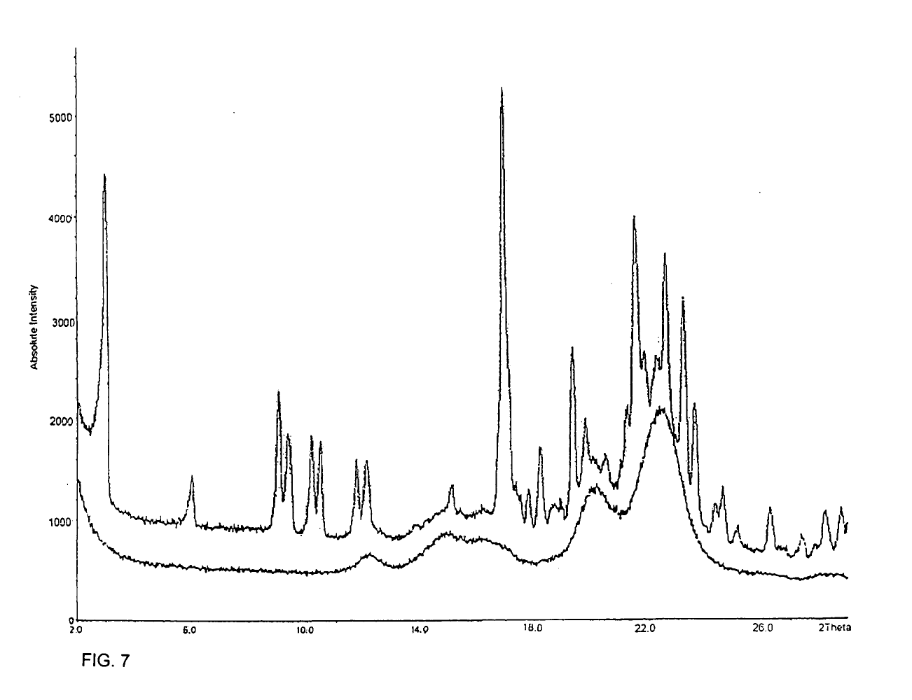

FIG. 8 a powder x-ray diffraction diagram of crystalline atorvastatin (so-called form V, cf. WO 01/36384 A1) in powder mixture ratio with microcrystalline cellulose of 1:1 as well as microcrystalline cellulose alone in the lower curve as comparison.

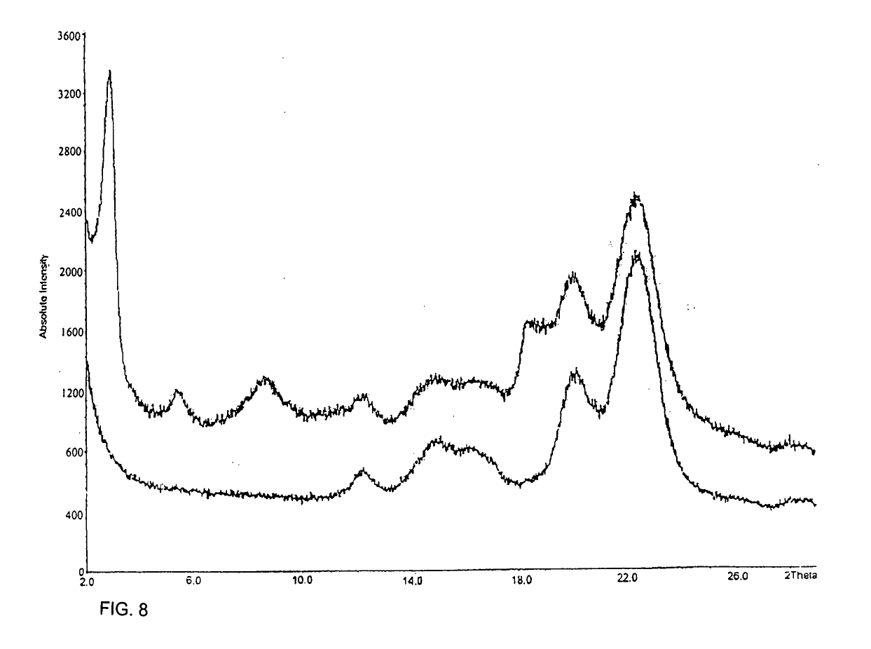

FIG. 9 an FTIR spectrum of an atorvastatin-microcrystalline cellulose adsorbate according to the invention (in the ratio of 1:1),

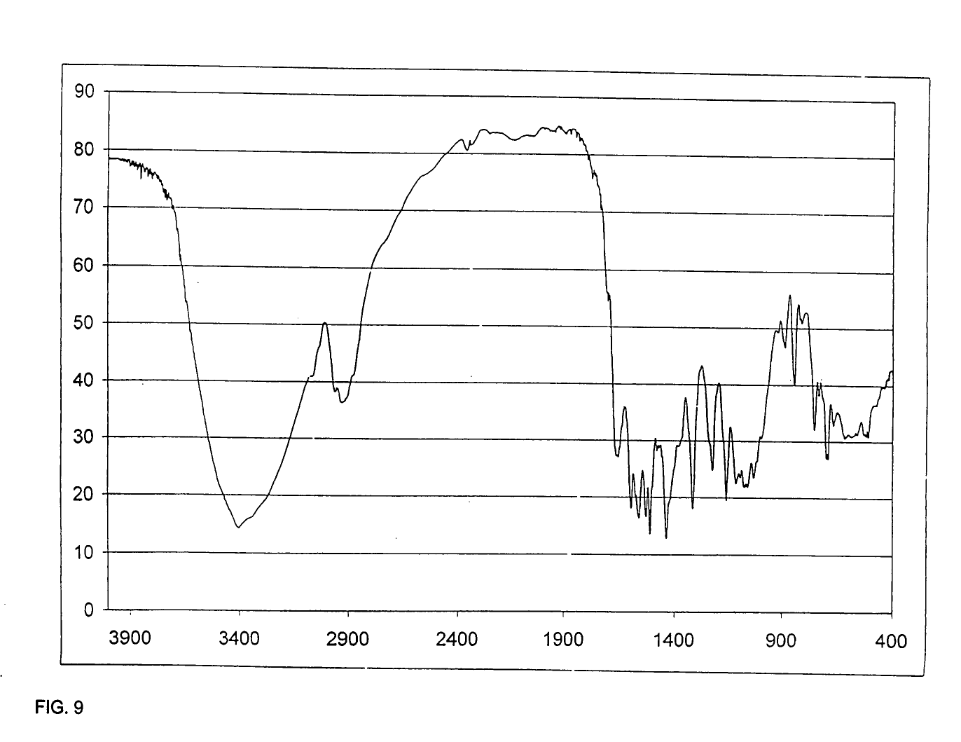

FIG. 10 an FTIR spectrum of an atorvastatin-mannitol adsorbate according to the invention (in the ratio of 1:1), and

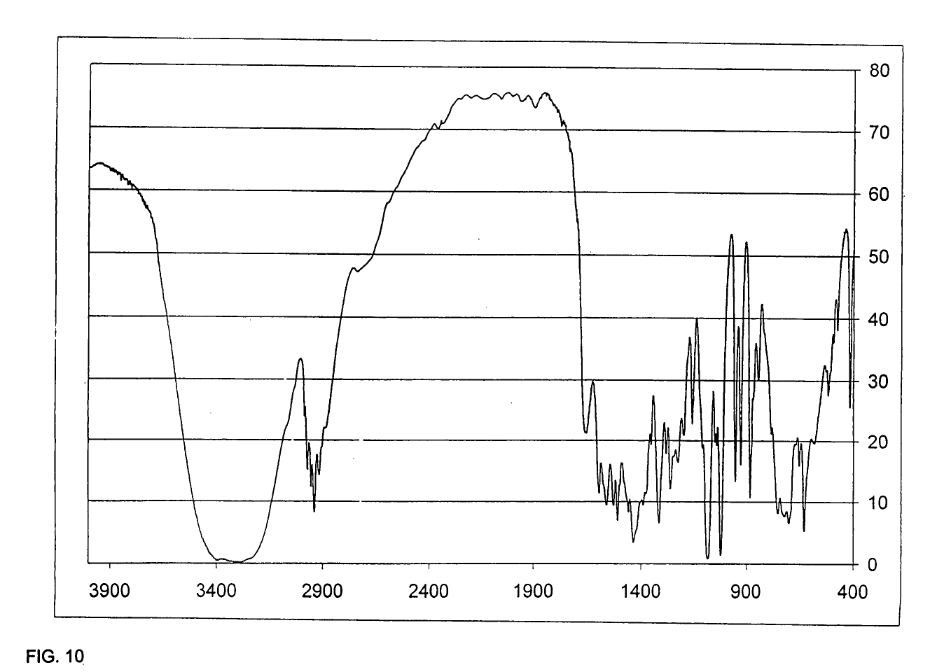

FIG. 11 an FTIR spectrum of a atorvastatin-lactose adsorbate according to the invention (in the ratio of 1:1).

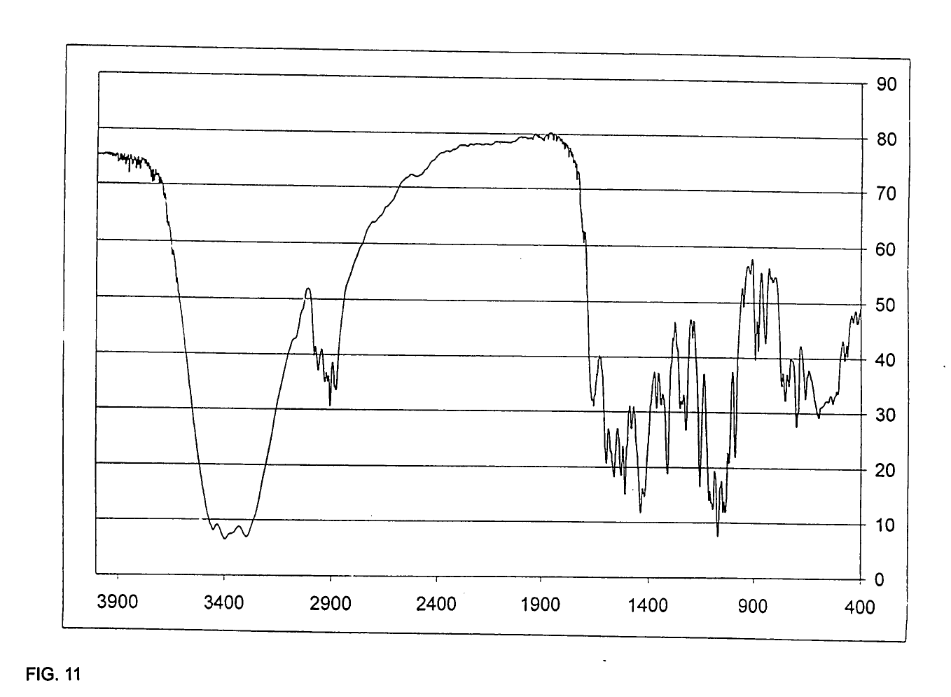

### EXAMPLES 1 TO 6

Used industrial equipment for the analytic studies:

HPLC Measurements:

All HPLC measurements were performed with an Agilent 100-HPLC.

| | |
|---|---|
| Column used: | Inertril ODS 2.5μ (150 × 4.6 mm) |
| Mobile phase: | 55% of 0.05 M sodium acetate |
| | 54% of acetonitrile |
| | pH: 4.0 (adjustment by acetic acid) |
| Flow rate: | 1 ml min⁻¹ |
| Detector: | UV at 246 nm |
| Injection volume: | 20 ml |
| Retention time atorvastatin: | approx. 15 min |
| Analysis duration: | 60 min |

X-Ray Measurements:

All powder x-ray diffraction diagrams were measured as follows:

| | |
|---|---|
| Appliance: | STADI P transmission diffractometer |
| | Cu-Kaₗ radiation (l = 1,54056 Å), U = 40 kV, I = 35 mA |
| | Primary ray monochromatic illuminator (crooked Ge 111) |
| Detector: | Linear position sensitive |
| Width of slit: | 1 mm |
| Linear PSD: | 2θ = 2° to 34°, 25 s/0.2° stepwise, increment Δ2θ = 0.02 |
| Sample: | Powder in mylar film |

IP Spectra:

| | |
|---|---|
| Appliance: | GENESIS II FTIR spectrometer |
| Measuring method: | KBr pressed part having 1% of test substance |

The spectra are shown as transmission values (in %) in dependence on the wave number (cm⁻¹).

### EXAMPLE 1

### Atorvastatin-Microcrvstalline Cellulose Adsorbate

To a solution of heterogeneous atorvastatin hemicalcium in acetone (0.15 g/mL) are added 0.15 g/mL of microcrystalline cellulose (CelphereSCP-100′) and uniformly suspended. Then, the solvent is dried up under permanent motion and vacuum (rotary evaporator or asymmetric moved dryer) at 25° C. Finally, the mixture is post-dried at 35° C. for a short time for removing residual solvent.

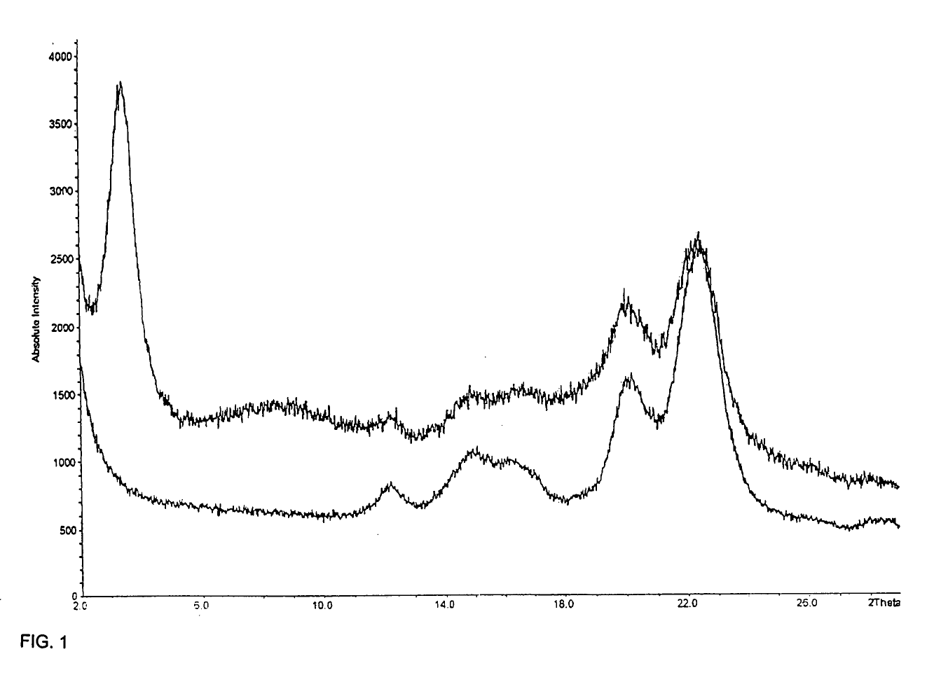

Active pharmaceutical ingredient amount of the adsorbate by means of HPLC: 49.6% (theoretically 50%)
Powder x-ray diffraction diagram: FIG. 1

Impurity profile: Sum of all the impurities: HPLC, in %:

| | Start | 15 days (70° C./75% relative humidity) |
|---|---|---|
| Sample (adsorbate) | 0.76 | 1.11 |
| Comparison (amorphous | 1.07 | 1.92 |
| atorvastatin calcium) | | |
| Tablet | 0.77 | 1.07 |

Atorvastatin tablets were produced from the adsorbate by direct pressing according to following composition:

| | |
|---|---|
| Atorvastatin-microcrystalline cellulose adsorbate | 80 mg |
| Microcrystalline cellulose (CelphereSCP-100 ®) | 408 mg |
| Adjuvants (Croscarmellose sodium, sodium laurylsulfate, | 72 mg |
| silica, magnesium stearate) in the usual amounts | |

The used amounts of the further adjuvants are known by those skilled in the art due to their basic knowledge and can be taken from standard works for formulating tablets, e.g. Ritschel et al., die Tablette, Editio Cantor—Aulendorf, 2. Auflage [2002].

Properties of the mixture ready for pressing and the tablet:

| | |
|---|---|
| Compressibility and flowability: | good |
| Medium hardness | 142 N |
| Attrition: | 0.06% (determined according |
| | to Ph. Eur.) |
| Decay period: | 40 sec. (determined according |
| | to Ph. Eur.) |
| Release: | 100% after 5 min. (Ph. Eur., |
| | 1000 mL water, |
| | 37° C., paddle 75 rpm) |

The so-obtained tablets can be provided with a coating, if required.

### EXAMPLE 2

### Atorvastatin-Mannitol Adsorbate

To a solution of heterogeneous atorvastatin hemicalcium in acetone (0.15 g/mL) are added 0.15 g/mL of mannitol (Mannogern®) and uniformly suspended. Then the solvent is dried up under permanent motion and vacuum (rotary evaporator or asymmetric moved dryer) at 25° C. Finally the mixture is post-dried at 35° C. for a short time for removing residual solvent.

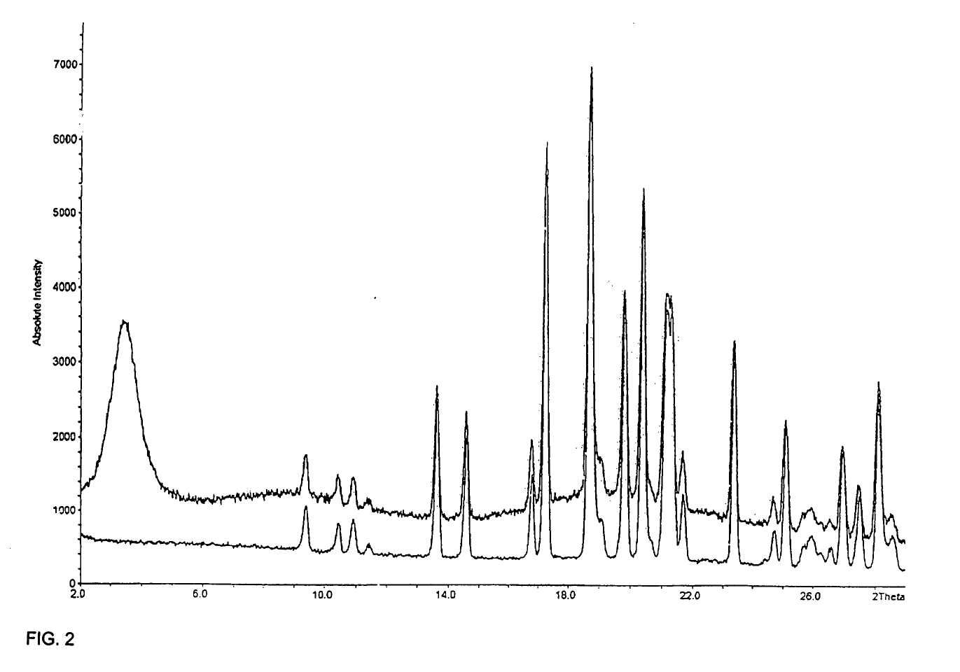

Active pharmaceutical ingredient amount of the adsorbate by means of HPLC: 49.85% (theoretically 50%)
Powder x-ray diffraction diagram: FIG. 2

Impurity profile (Sum of all the impurities, HPLC, in %):

| | Start | 15 days (70° C./75% relative humidity) |
|---|---|---|
| Sample (adsorbate) | 0.91 | 1.40 |
| Comparison (amorphous | 1.07 | 1.92 |
| atorvastatin calcium) | | |
| Tablet | 0.85 | 1.01 |

Atorvastatin tablets were produced from the adsorbate by direct pressing according to following composition:

| | |
|---|---|
| Atorvastatin-mannitol adsorbate | 80 mg |
| Mannitol | 408 mg |
| Adjuvants (as in Ex. 1) | 72 mg |

Properties of the mixture ready for pressing and the tablets:

| | |
|---|---|
| Compressibility and flowability: | satisfactory till good |
| Medium hardness | 153 N |
| Attrition: | 0.18% (determined according |
| | to Ph. Eur.) |
| Decay period: | 65 sec. (determined according |
| | to Ph. Eur.) |
| Release: | 100% after 7 min. (Ph. Eur., |
| | 1000 mL water, |
| | 37° C., paddle 75 rpm) |

The so-obtained tablets can be provided with a coating, if required.

### EXAMPLE 3

### Atorvastatin-Lactose Adsorbate

To a solution of heterogeneous atorvastatin hemicalcium in acetone (0.15 g/mL) are added 0.15 g/mL of lactose (Lactopress® Anhydrous) and uniformly suspended. Then the solvent is dried up under permanent motion and vacuum (rotary evaporator or asymmetric moved dryer) at 25° C. Finally, the mixture is post-dried at 35° C. for a short time for removing residual solvent.

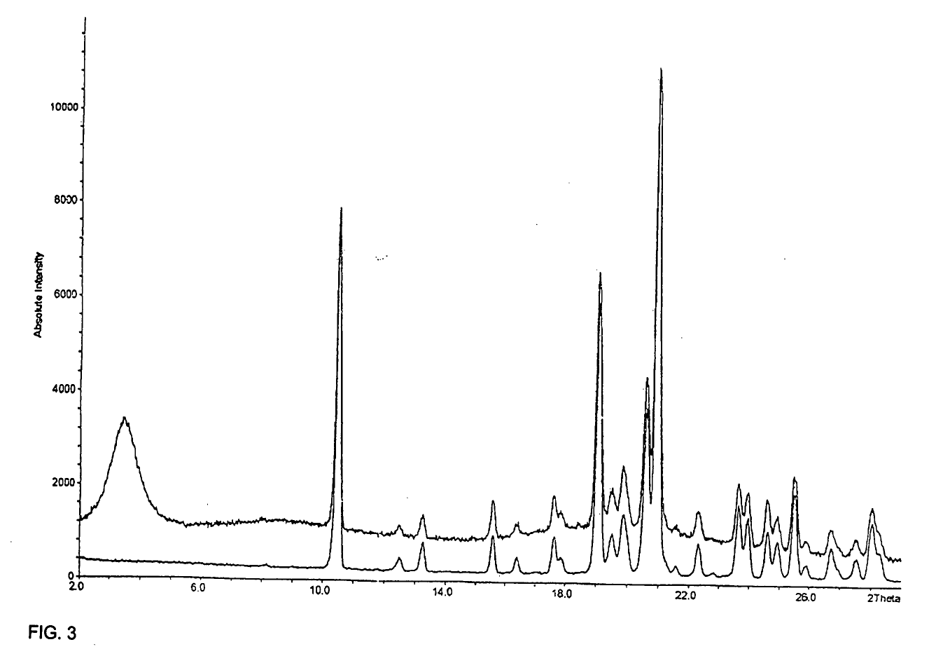

Active pharmaceutical ingredient amount of the adsorbate by means of HPLC: 51.18% (theoretically 50%)
Powder x-ray diffraction diagram: FIG. 3

Impurity profile (Sum of all the impurities, HPLC, in %):

| | Start | 15 days (70° C./75% relative humidity) |
|---|---|---|
| Sample (adsorbate) | 0.80 | 1.03 |
| Comparison (amorphous | 1.07 | 1.92 |
| atorvastatin calcium) | | |
| Tablet | 0.79 | 1.05 |

Atorvastatin tablets were produced from the adsorbate by direct pressing according to following composition:

| | |
|---|---|
| Atorvastatin-lactose adsorbate | 80 mg |
| Lactose | 408 mg |
| Adjuvants (as in Ex. 1) | 72 mg |

Properties of the mixture ready for pressing and the tablets:

| | |
|---|---|
| Compressibility and flowability: | satisfactory till good |
| Medium hardness | 138 N |
| Attrition: | 0.18% (determined according |
| | to Ph. Eur.) |
| Decay period: | 80 sec. (determined according |
| | to Ph. Eur.) |
| Release: | 100% after 8 min. (Ph. Eur., |
| | 1000 mL water, |
| | 37° C., paddle 75 rpm) |

The so-obtained tablets can be provided with a coating, if required.

### EXAMPLE 4

### Atorvastatin-Microcrystalline Cellulose Adsorbate

To a solution of heterogeneous atorvastatin hemicalcium in ethanol (0.15 g/mL) are added 0.225 g/mL of microcrystalline cellulose (ratio of active pharmaceutical ingredient: adsorbate 2:3) and uniformly suspended. Then the solvent is dried up under permanent motion and vacuum (rotary evaporator or asymmetric moved dryer) at 25° C. Finally, the mixture is post-dried at 35° C. for a short time for removing residual solvent.

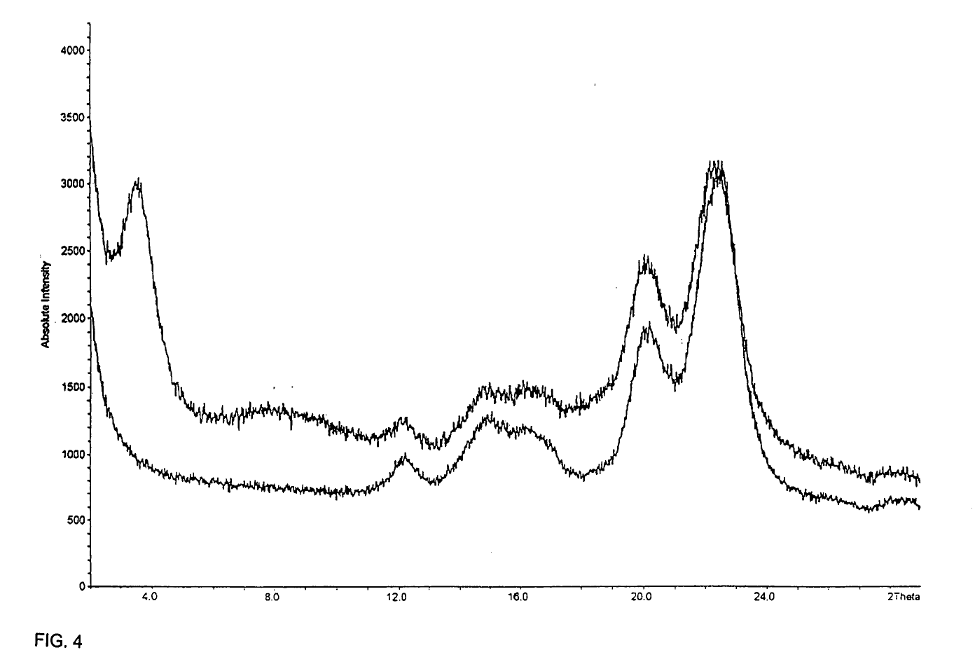

Active pharmaceutical ingredient amount of the adsorbate by means of HPLC: 40.3% (theoretically 40%)
Powder x-ray diffraction diagram: FIG. 4

Impurity profile (Sum of all the impurities, HPLC, in %):

| | Start | 15 days (70° C./75% relative humidity) |
|---|---|---|
| Sample (adsorbate) | 0.83 | 1.04 |
| Comparison (amorphous | 1.07 | 1.92 |
| atorvastatin calcium) | | |

### EXAMPLE 5

### Atorvastatin-Mannitol Adsorbate

To a solution of heterogeneous atorvastatin hemicalcium in ethanol (0.15 g/mL) are added 0.15 g/mL of mannitol (1:1 mixture) and uniformly suspended. Then the solvent is dried up under permanent motion and vacuum (rotary evaporator or asymmetric moved dryer) at 25° C. Finally, the mixture is post-dried at 35° C. for a short time for removing residual solvent.

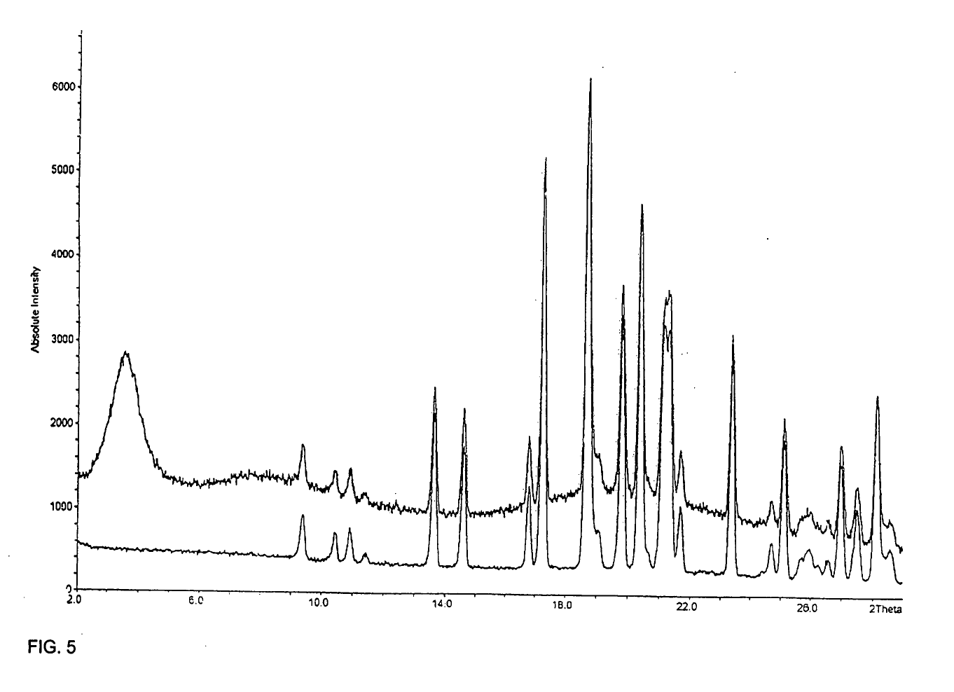

Active pharmaceutical ingredient amount of the adsorbate by means of HPLC: 50.4% (theoretically 50%)
Powder x-ray diffraction diagram: FIG. 5

Impurity profile (Sum of all the impurities, HPLC, in %):

| | Start | 15 days (70° C./75% relative humidity) |
|---|---|---|
| Sample (adsorbate) | 0.72 | 1.05 |
| comparison (amorphous | 1.07 | 1.92 |
| atorvastatin calcium) | | |

### EXAMPLE 6

### Atorvastatin-Microcrystalline Cellulose Adsorbate

To a solution of heterogeneous atorvastatin hemicalcium in ethanol (0.15 g/mL) are added 0.30 g/mL of microcrystalline cellulose (ratio of active pharmaceutical ingredient:adsorbate 1:2) and uniformly suspended. Then the solvent is dried up under permanent motion and vacuum (rotary evaporator or asymmetric moved dryer) at 25° C. Finally, the mixture is post-dried at 35° C. for a short time for removing residual solvent.

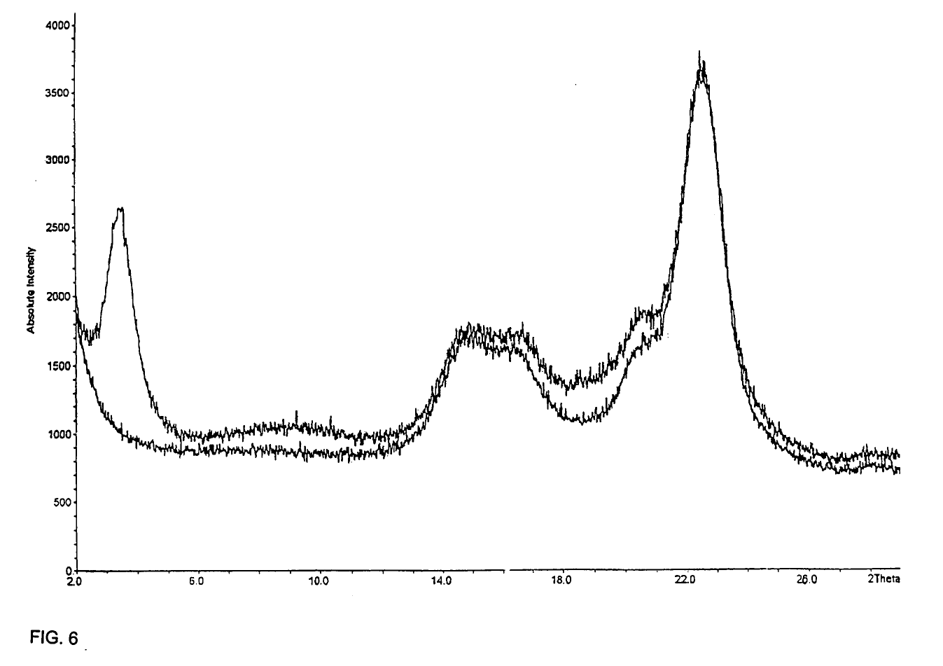

Active pharmaceutical ingredient amount of the adsorbate by means of HPLC: 33.4% (theoretically 33.3%)
Powder x-ray diffraction diagram: FIG. 6

Impurity profile (Sum of all the impurities, HPLC, in %):

| | Start | 15 days (70° C./75% relative humidity) |
|---|---|---|
| Sample (adsorbate) | 0.91 | 1.09 |
| comparison (amorphous | 1.07 | 1.92 |
| atorvastatin calcium) | | |

1. A method for the preparation of atorvastatin adsorbates and solvates thereof, comprising the steps:
starting from a solution of the atorvastatin or one of its salts, hydrates, solvates, esters, lactones and tautomeric derivatives thereof in at least one organic solvent having a total water content of not more than 10 vol.-%, preferably not more than 5 vol.-%,
suspending therein an adsorber material selected from the group consisting of celluloses, cellulose derivatives, polyols, sugars, sugar derivatives, maltodextrins, cyclodextrins, starches, polydextroses or mixtures thereof, and
removing the solvent by drying.

2. The method according to claim 1, characterized in that the adsorber material is selected from the group of microcrystalline cellulose, lactose and mannitol.

3. The method according to claim 1, characterized in that the atorvastatin adsorbates comprise the active pharmaceutical ingredient in finely dispersed amorphous form, and if required, as the solvates thereof.

4. The method according to claim 1, characterized in that one adjusts a ratio of active pharmaceutical ingredient to adsorbate is adjusted in the range of from 1:0.1 to 10, especially in the range of from 1:1 to 1:2.

5. The method according to claim 1, characterized in that organic solvents having a total water amount of not more than 10 vol.-%, alone or in mixtures, as solvents are used, wherein the organic solvents are selected from the group of the lower alkanols having 1 to 4 carbon atoms, the group of the ethers and the group of the aliphatic ketones and mixtures thereof.

6. The method according to claim 5, characterized in that acetone, ethanol, methanol, methyl ethyl ketone or mixtures thereof as solvents are used.

7. The method according to claim 1, characterized in that one uses an active pharmaceutical ingredient solution which arises within the scope of the atorvastatin synthesis and suspends the adsorber material therein is used.

8. Atorvastatin adsorbates and solvates thereof obtained according to the method of claim 1.

9. Atorvastatin adsorbates and solvates thereof obtained according to the method of claim 2.

10. Atorvastatin adsorbates and solvates thereof obtained according to the method of claim 3.

11. Atorvastatin adsorbates and solvates thereof obtained according to the method of claim 4.

12. Atorvastatin adsorbates and solvates thereof obtained according to the method of claim 5.

13. Atorvastatin adsorbates and solvates thereof obtained according to the method of claim 6.

14. Atorvastatin adsorbates and solvates thereof obtained according to the method of claim 7.

15. Atorvastatin adsorbates and solvates thereof, characterized in that the atorvastatin adsorbate comprises the active pharmaceutical ingredient in a finely dispersed amorphous form, preferably as alkali metal salt or as alkaline earth metal salt as well as the hydrates and solvates thereof and especially preferred as calcium salt as well as the hydrates thereof.

16. A pharmaceutical formulation having at least one active pharmaceutical ingredient, and if required, further pharmaceutically acceptable adjuvants, characterized in that the atorvastatin adsorbates according to claim 8 are used as active pharmaceutical ingredient.

17. A pharmaceutical formulation having at least one active pharmaceutical ingredient, and if required, further pharmaceutically acceptable adjuvants, characterized in that the atorvastatin adsorbates according to claim 9 are used as active pharmaceutical ingredient.

18. A pharmaceutical formulation having at least one active pharmaceutical ingredient, and if required, further pharmaceutically acceptable adjuvants, characterized in that the atorvastatin adsorbates according to claim 10 are used as active pharmaceutical ingredient.

19. A pharmaceutical formulation having at least one active pharmaceutical ingredient, and if required, further pharmaceutically acceptable adjuvants, characterized in that the atorvastatin adsorbates according to claim 11 are used as active pharmaceutical ingredient.

20. A pharmaceutical formulation having at least one active pharmaceutical ingredient, and if required, further pharmaceutically acceptable adjuvants, characterized in that the atorvastatin adsorbates according to claim 12 are used as active pharmaceutical ingredient.

21. A pharmaceutical formulation having at least one active pharmaceutical ingredient, and if required, further pharmaceutically acceptable adjuvants, characterized in that the atorvastatin adsorbates according to claim 13 are used as active pharmaceutical ingredient.

22. A pharmaceutical formulation having at least one active pharmaceutical ingredient, and if required, further pharmaceutically acceptable adjuvants, characterized in that the atorvastatin adsorbates according to claim 14 are used as active pharmaceutical ingredient.

23. A pharmaceutical formulation having at least one active pharmaceutical ingredient, and if required, further pharmaceutically acceptable adjuvants, characterized in that the atorvastatin adsorbates according to claim 15 are used as active pharmaceutical ingredient.

24. The pharmaceutical formulation according to claim 16, characterized in that it exists in form of tablets, capsules, pellets and granulates which are obtainable in a known manner by usual pharmaceutically acceptable adjuvants.

25. The pharmaceutical formulation according to claim 16 in form of tablets which rapidly release the active pharmaceutical ingredient and are produced by direct pressing.

26. The pharmaceutical formulation according to claim 24 in form of tablets which rapidly release the active pharmaceutical ingredient and are produced by direct pressing.
